(19) United States
(12) Patent Application Publication
(10) Pub. No.: US 2009/0318686 A1
(43) Pub. Date: Dec. 24, 2009

(54) METHOD FOR PRODUCING POTASSIUM SALT OF 3,4-DIHYDRO-1,2,3-OXATHIAZIN-4-ONE-2,2-DIOXIDE COMPOUND

(76) Inventors: Akira Saito, Niigata (JP); Atsushi Mori, Niigata (JP)

Correspondence Address: BIRCH STEWART KOLASCH & BIRCH, PO BOX 747, FALLS CHURCH, VA, 22040-0747, US

(21) Appl. No.: 12/373,927

(22) PCT Filed: Aug. 2, 2007
(86) PCT No.: PCT/JP2007/065137

§ 371 Date: Jan. 15, 2009

(30) Foreign Application Priority Data
Aug. 3, 2006 (JP) 2006-212262

(51) Int. Cl. C07D 291/06

(52) U.S. Cl. 544/2

(57) ABSTRACT

A method for producing a high quality potassium salt of 3,4-dihydro-1,2,3-oxathiazin-4-one-2,2-dioxide compound easily and efficiently is provided.

The producing method of the invention includes the steps of neutralizing 3,4-dihydro-1,2,3-oxathiazin-4-one-2, 2-dioxide compound represented by the following formula (1)

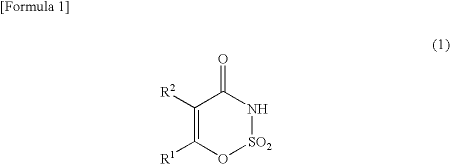

(wherein R¹ and R² are the same as or different from each other and are hydrogen atom or an organic group inert to the reaction) with potassium hydroxide in a mixed solvent of water and a water soluble organic solvent to form a potassium salt of 3,4-dihydro-1,2,3-oxathiazin-4-one-2,2-dioxide compound represented by the following formula (2)

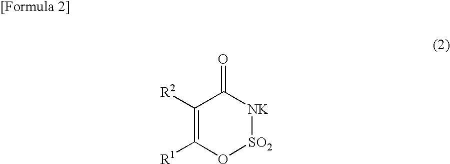

and, during the neutralization, precipitating potassium sulfate derived from sulfuric acid contained in the compound represented by Formula (1) as an impurity.

### TECHNICAL FIELD

The present invention relates to a method for producing potassium salts of 3,4-dihydro-1,2,3-oxathiazin-4-one-2,2-dioxide compounds which are useful as sweeteners or raw materials therefor in food industry or intermediate materials for fine chemicals or the like.

### BACKGROUND ART

As for a method for producing a potassium salt of 3,4-dihydro-1,2,3-oxathiazin-4-one-2,2-dioxide compound, Patent Documents 1 to 3 disclose the method in which acetoacetamide-N-sulfonic acid or a salt thereof is reacted with sulfuric anhydride (SO₃) in an inert organic solvent to cyclize and ring-close, then the product is hydrolyzed to obtain a 6-methyl-3,4-dihydro-1,2,3-oxathiazin-4-one-2,2-dioxide compound, and the obtained compound is neutralized with potassium hydroxide. As for methods for separating and purifying the formed potassium salt of 3,4-dihydro-1,2,3-oxathiazin-4-one-2,2-dioxide compound, Japanese Unexamined Patent Publication No. 62-56481 discloses the following methods: (1) evaporating and concentrating a liquid in an organic phase after the hydrolysis to obtain a residue, dissolving the residue in methanol and reacting it with potassium hydroxide in methanol to precipitate the potassium salt of 3,4-dihydro-1,2,3-oxathiazin-4-one-2,2-dioxide compound at once, and filtering and drying the precipitate for isolation; (2) mixing a liquid in an organic phase after the hydrolysis with a dilute aqueous solution of potassium hydroxide, stirring the mixture, concentrating and cooling the aqueous phase liquid-separated from the mixture to precipitate the potassium salt of 3,4-dihydro-1,2,3-oxathiazin-4-one-2,2-dioxide compound, and filtering and drying the precipitate for isolation; and (3) mixing a liquid in an organic phase after the hydrolysis with an aqueous solution of potassium hydroxide in high concentration, stirring the mixture to precipitate a potassium salt of 3,4-dihydro-1,2,3-oxathiazin-4-one-2,2-dioxide compound, and filtering and drying the potassium salt for isolation.

However, in the method using sulfuric anhydride (SO₃) as a cyclizing agent, sulfuric acid is formed as a by-product during the hydrolysis after the cyclization, so that the formed sulfuric acid is mixed in the 6-methyl-3,4-dihydro-1,2,3-oxathiazin-4-one-2,2-dioxide compound as an impurity. The formed sulfuric acid is converted to potassium sulfate during the neutralization with potassium hydroxide. The difference in solubility between the potassium salt of 3,4-dihydro-1,2,3-oxathiazin-4-one-2,2-dioxide compound and potassium sulfate in water or methanol is not so large, so that, as disclosed in Japanese Unexamined Patent Publication No. 62-56481, when the potassium salt of 3,4-dihydro-1,2,3-oxathiazin-4-one-2,2-dioxide compound is crystallized from water or methanol, potassium sulfate is mixed in the product and deteriorate the quality.

Patent Document 1: Japanese Unexamined Patent Publication No. 62-56481

Patent Document 2: Japanese Unexamined Patent Publication No. 62-129277

Patent Document 3: Japanese Unexamined Patent Publication No. 2005-263779

### DISCLOSURE OF THE INVENTION

### Technical Problems to be Solved

An object of the present invention is to provide a method for producing a high quality potassium salt of 3,4-dihydro-1,2,3-oxathiazin-4-one-2,2-dioxide compound easily and efficiently.

Another object of the invention is to provide a method for producing a potassium salt of 3,4-dihydro-1,2,3-oxathiazin-4-one-2,2-dioxide compound with less potassium sulfate content easily and efficiently.

### Means to Solve the Problems

After intensive investigations to achieve the above object, the present inventors have found that the difference in solubility between the potassium salt of 3,4-dihydro-1,2,3-oxathiazin-4-one-2,2-dioxide compound and potassium sulfate is not so large in a single solvent such as water and methanol, but in a mixed solvent of water and a water soluble organic solvent, the difference in solubility is large, and thus, when a water soluble organic solvent is added to the reaction mixture after a reaction of the 3,4-dihydro-1,2,3-oxathiazin-4-one-2,2-dioxide compound with potassium hydroxide in a mixed solvent of water and a water soluble organic solvent, or after a reaction of the 3,4-dihydro-1,2,3-oxathiazin-4-one-2,2-dioxide compound with potassium hydroxide in water or in a mixed solvent of water and a water soluble organic solvent, a precipitate of potassium sulfate derived from sulfuric acid contained in the 3,4-dihydro-1,2,3-oxathiazin-4-one-2,2-dioxide compound as the impurity should form first from the liquid containing the potassium salt of 3,4-dihydro-1,2,3-oxathiazin-4-one-2,2-dioxide compound in the dissolving state, and have completed the present invention.

Specifically, the present invention provides a method for producing a potassium salt of 3,4-dihydro-1,2,3-oxathiazin-4-one-2,2-dioxide compound represented by the following formula (2).

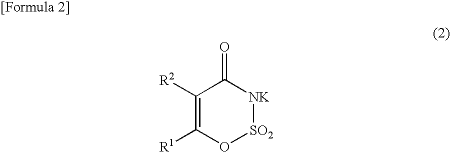

In the formula (2), R¹ and R² are the same as or different from each other and are hydrogen atom or an organic group inert to the reaction. The potassium salt of 3,4-dihydro-1,2,3-oxathiazin-4-one-2,2-dioxide compound contains sulfuric acid as an impurity. The method comprises steps of: neutralizing 3,4dihydro-1,2,3-oxathiazin-4-one-2,2-dioxide compound represented by the following formula (1)

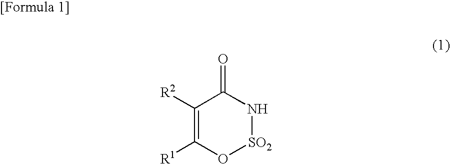

(wherein R¹ and R²are as defined above) with potassium hydroxide in a mixed solvent of water and a water soluble organic solvent to obtain a solution containing said potassium salt; and precipitating potassium sulfate derived from the sulfuric acid upon taking the step of neutralizing.

Further, the present invention provides a method for producing a potassium salt of 3,4-dihydro-1,2,3oxathiazin-4-one-2,2-dioxide compound represented by the following formula (2).

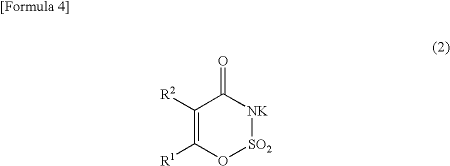

In the formula (2), R¹ and R² are the same as or different from each other and are hydrogen atom or an organic group inert to the reaction. The potassium salt of 3,4-dihydro-1,2,3-oxathiazin-4-one-2,2-dioxide compound contains sulfuric acid as an impurity. The method comprises steps of: neutralizing 3,4-dihydro-1,2,3-oxathiazin-4-one-2,2-dioxide compound represented by the following formula (1)

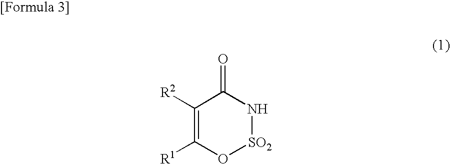

(wherein R¹ and R²are as defined above) with potassium hydroxide in a solvent containing at least water to obtain an aqueous solution containing said potassium salt; and precipitating potassium sulfate derived from the sulfuric acid by adding a water soluble organic solvent into the aqueous solution containing said potassium salt after the step of neutralizing.

Preferably, the water soluble organic solvent is methanol in the above-mentioned methods.

Preferably, the methods further comprise a step of separating and removing the precipitated potassium sulfate by filtration, and a step of crystallizing the potassium salt of 3,4-dihydro-1,2,3-oxathiazin-4-one-2,2-dioxide compound from the solution after removing the precipitated potassium sulfate. When the methods comprise the latter step, the solution after separating and removing the crystallized potassium salt of 3,4-dihydro-1,2,3-oxathiazin-4-one-2,2-dioxide compound may be recycled to the step of neutralizing.

### ADVANTAGES OF THE INVENTION

According to the present invention, 3,4-dihydro-1,2,3-oxathiazin-4-one-2,2-dioxide compound is neutralized with potassium hydroxide in a mixed solvent of water and a water soluble organic solvent, or, after the neutralization of 3,4-dihydro-1,2,3-oxathiazin-4-one-2,2-dioxide compound with potassium hydroxide, a water soluble organic solvent is added to the reaction mixture. Accordingly, potassium sulfate as an impurity is precipitated prior to the potassium salt of 3,4-dihydro-1,2,3-oxathiazin-4-one-2,2-dioxide compound. Therefore, a high quality potassium salt of 3,4-dihydro-1,2,3-oxathiazin-4-one-2,2-dioxide compound with less potassium sulfate content may be obtained easily and efficiently.

### BRIEF DESCRIPTION OF DRAWINGS

FIG. 1 is a graph showing relations between the amount of water/methanol in a water-methanol mixed solvent (methanol concentration (%)) and the solubilities of potassium salt of 6-methyl-3,4-dihydro-1,2,3-oxathiazin-4-one-2,2-dioxide (ASK) and potassium sulfate (K₂SO₄) at 40° C. in the solvent (concentration (%) in saturated solution).

FIG. 2 is a graph showing relations between the solubilities of ASK in various solvents (concentration (%) in saturated solution) and temperature.

FIG. 3 is a graph showing relations between the solubilities of potassium sulfate (K₂SO₄) in various solvents (concentration (%) in saturated solution) and temperature.

### BEST MODE FOR CARRYING OUT THE INVENTION

The first producing method of the present invention includes the steps of neutralizing 3,4-dihydro-1,2,3-oxathiazin-4-one-2,2-dioxide compound represented by Formula (1) with potassium hydroxide in a mixed solvent of water and a water soluble organic solvent to form a potassium salt of 3,4-dihydro-1,2,3-oxathiazin-4-one-2,2-dioxide compound represented by Formula (2), and precipitating potassium sulfate derived from sulfuric acid contained in the compound represented by Formula (1) as an impurity during the neutralization. Furthermore, the second producing method of the present invention includes the steps of neutralizing 3,4-dihydro-1,2,3-oxathiazin-4-one-2,2-dioxide compound represented by Formula (1) with potassium hydroxide in a solvent containing at least water to form a potassium salt of 3,4-dihydro-1,2,3-oxathiazin-4-one-2,2-dioxide compound represented by Formula (2), and adding a water soluble organic solvent into the aqueous solution containing the potassium salt represented by Formula (2) after the neutralization to precipitate potassium sulfate derived from sulfuric acid contained in the compound represented by Formula (1) as an impurity.

In Formulae (1) and (2), R¹ and R² are the same as or different from each other and are hydrogen atom or an organic group inert to the reaction. The organic group inert to the reaction is not particularly limited, as long as being the organic group that is inert (inactive) under the reaction conditions. Examples are alkyl groups, alkenyl groups, alkynyl groups, cycloalkyl groups, acyl groups, aralkyl groups, aryl groups and the like. The alkyl groups include straight or branched chain C₁₋₁₀ alkyl groups (for example, C₁₋₆ alkyl groups such as methyl group, ethyl group, propyl group, butyl group, isobutyl group, and tert-butyl group). The alkenyl groups include straight or branched chain C₂₋₁₀ alkenyl groups (for example, C₂₋₅ alkenyl groups such as vinyl group, allyl group, isopropenyl group, 1-butenyl group, and 2-butenyl group). The alkynyl groups include straight or branched chain C₂₋₁₀ alkynyl groups (for example, C₂₋₅ alkynyl groups such as ethynyl group, propynyl group, 1-butynyl group, and 2-butynyl group). The cycloalkyl groups include, for example, C₃₋₁₀ cycloalkyl groups such as cyclopropyl group, cyclobutyl group, cyclopentyl group, and cyclohexyl group (preferably, C₄₋₈ cycloalkyl groups). The acyl groups include straight or branched chain C₂₋₁₀ aliphatic acyl groups (for example, acetyl group, propionyl group, butyryl group, isobutyryl group, and valeryl group), and C₇₋₁₁ aromatic acyl groups (for example, benzoyl group, toluyl group, and naphthoyl group). The aralkyl groups include C₆₋₁₀ aryl-C₁₋₄ alkyl groups (for example, benzyl group) and the like, and the aryl groups include C₆₋₁₀ aryl groups such as phenyl group, and the like.

In Formulae (1) and (2), R¹ and R² may be composed of any suitable combination, and for example, a combination in which R¹ and R² are each hydrogen atom or a C₁₋₄ alkyl group is preferred. Among them, as for the compound represented by Formula (1), a compound in which R¹ is a C₁₋₄ alkyl group and R² is hydrogen atom is preferred, and specifically, the compound in which R¹ is methyl group and R² is hydrogen atom is preferred.

3,4-dihydro-1,2,3-oxathiazin-4-one-2,2-dioxide compound represented by Formula (1) may be obtained, for example, by cyclization of β-ketoamide-N-sulfonic acid represented by Formula (3):

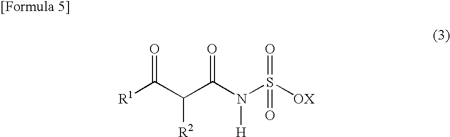

(wherein R¹ and R² are as defined above and X is hydrogen atom) or a salt thereof under the presence of acid anhydride, or further hydrolysis of the product.

The salt of β-ketoamide-N-sulfonic acid compound represented by Formula (3) includes salts (sulfonates) in which the sulfonic group is neutralized with a base, and salts in which the —NH— group in the formula is neutralized with a base. Examples of these salts (salts of sulfonic acid, and salts of —NH—) are metal salts, ammonium salts, salts of organic bases and the like. Examples of the metal salts are salts of alkali metals (Group 1A metals of the Periodic Table) such as Li, Na, and K; salts of alkaline earth metals (Group 2A metals of the Periodic Table) such as Mg, Ca, Sr, and Ba; salts of metals of Group 3B of the Periodic Table such as Al and Ga; and salts of transition metals (for example, Group 3A metals, Group 4A metals, Group 5A metals, Group 6A metals, Group 7A metals such as Mn, Group 8 metals such as Fe, Group 1B metals such as Cu, Ag, and Au, Group 2B metals such as Zn, Group 4B metals, and Group 5B metals of the Periodic Table). Preferred metal salts include salts of mono-, di-, or tri-valent metals, for example, salts of alkali metals (Na, K, and the like), salts of alkaline earth metals (Mg, Ca, and the like), Al salts, and salts of transition metals (Mn, Fe, and the like), and the like. In consideration of economical efficiency, safety and the like, salts of alkali metals such as Na and K are specifically preferred.

Examples of the organic bases are aliphatic amines [primary amines (for example, C₁₋₁₀ monoalkylamines such as methylamine and ethylamine), secondary amines (for example, di-C₁₋₁₀ alkylamines such as dimethylamine and ethylmethylamine), and tertiary amines (for example, tri-C₁₋₁₀ alkylamines such as trimethylamine and triethylamine)], alicyclic amines (for example, mono-, di-, or tri-C₃₋₁₂ cycloalkylamines such as cyclohexylamine), aromatic amines (for example, mono-C₆₋₁₀ arylamines such as aniline and dimethylaniline, di-C₆₋₁₀ arylamines such as diphenylamine, tri-C₆₋₁₀ arylamines such as triphenylamine, and aralkylamines such as benzylamine), cyclic amines (for example, piperidine, N-methylpiperidine, and morpholine), nitrogen-containing aromatic heterocyclic compounds (for example, pyridine, quinoline, and derivatives thereof), and the like. Preferred organic bases include aliphatic amines. Furthermore, not only aliphatic amines but also any tertiary amine is preferred.

As for a salt of β-ketoamide-N-sulfonic acid compound represented by Formula (3) (sulfonate) a salt with a tertiary amine is specifically preferred.

The acid anhydride works as a cyclizing agent (cyclization-dehydration agent and the like) for β-ketoamide-N-sulfonic acid represented by Formula (3) or a salt thereof (hereinafter, sometimes simply referred to as “substrate”). Examples of the acid anhydride are acid anhydrides formed from inorganic acids such as sulfuric acid, halogenated sulfuric acids (fluorosulfuric acid, chlorosulfuric acid, and the like), pyrophosphoric acids (pyrophosphoric acid; halogenated pyrophosphoric acids such as fluoropyrophosphoric acid; and the like), nitric acid, and boric acid (orthoboric acid, metaboric acid, and the like); and formed from organic acids such as sulfonic acids, organic phosphoric acids (C₁₋₄ alkyl-phosphoric acids such as methylphosphoric acid; phosphoric acid mono-C₁₋₄ alkyl esters such as phosphoric acid monomethyl ester and phosphoric acid monoethyl ester) and the like. The acid anhydride may be any of an acid anhydride formed from elimination of water from one molecule of an acid, an acid anhydride formed from elimination of water from two or more molecules of an acid, and an acid anhydride formed from elimination of water from two or more molecules of different acids (mixed acid anhydride), and the like. The acid anhydrides may be used alone or as a mixture of two or more kinds of acid anhydrides. Preferred acid anhydride is the acid anhydride formed from an acid containing sulfuric acid, and sulfuric anhydride (SO₃) is specifically preferred.

The amount of the acid anhydride is generally at least 1 mol or more (for example, from about 1 to about 20 mol), preferably from about 1 to about 10 mol, and specifically preferably from about 4 to about 8 mol per 1 mol of the substrate.

The cyclization (cyclization-dehydration and the like) of β-ketoamide-N-sulfonic acid represented by Formula (3) or a salt thereof is generally carried out under the presence of a solvent. As for the reaction solvent, various inorganic and organic solvents inert to the reaction (specifically, not reacting with acid anhydride) may be used, but generally an organic solvent inert to the reaction is used. Furthermore, as for the reaction solvent, generally, a solvent containing substantially no water is used.

Examples of the organic solvent are aliphatic hydrocarbons (for example, pentane, hexane, and octane), alicyclic hydrocarbons (for example, cyclohexane), aromatic hydrocarbons (for example, benzene, toluene, xylene, and ethylbenzene), halogenated hydrocarbons (for example, haloalkanes such as dichloromethane, dichloroethane, chloroform, trichloroethylene, tetrachloroethylene, and trichlorofluoroethylene), esters (for example, carboxylic esters such as methyl acetate, ethyl acetate, butyl acetate, and methyl propionate), ketones (for example, aliphatic ketones such as acetone, methyl ethyl ketone, and methyl isobutyl ketone; and cyclic ketones such as cyclohexanone), ethers (for example, chain ethers such as diethyl ether, diisopropyl ether, 1,2-dimethoxyethane, Cellosolve, Carbitol, Diglyme, and diethylene glycol dimethyl ether; aromatic ethers such as anisole, 1,2-dimethoxybenzene, and diphenyl ether; and cyclic ethers such as tetrahydrofuran, dioxolane, and dioxane), sulfoxides (for example, dimethyl sulfoxide, sulfolane, 2-methylsulfolane, and 3-methylsulfolane), and the like. These solvents may be used alone or as A mixture of two or more kinds of solvents. Preferred solvents are halogenated hydrocarbons, and specifically preferably dichloromethane is used.

Preferably, the cyclization is continuously carried out using a continuous flow reactor. A tubular reactor or a motionless mixer is preferably used as the continuous flow reactor. In order to get a better result of the cyclization, it is preferred that the substrate and the acid anhydride [sulfuric anhydride (SO₃) and the like], to be reacted, are dissolved or dispersed in the solvent, respectively, and are cooled to, for example, 10° C. or below (from about −100° C. to about 10° C.), preferably from −80° C. to 10° C., and specifically preferably from −30° C. to 10° C. before the reaction. The concentration of the substrate in the mixture containing the substrate for feeding into the reactor may be appropriately selected in a range not deteriorating operability and the like, and is generally from about 0.1% to about 50% by weight, preferably from about 0.5% to about 30% by weight, and more preferably from about 1% to about 20% by weight (specifically from about 5% to about 15% by weight). The concentration of the acid anhydride in the mixture containing the acid anhydride [sulfuric anhydride (SO₃) and the like], to be fed into the reactor, may be appropriately selected in a range not deteriorating operability and the like, and is generally from about 0.1% to about 50% by weight, preferably from about 0.5% to about 30% by weight, and more preferably from about 5% to about 20% by weight.

The total used amount of the reaction solvent may be appropriately selected in consideration of reactivity, operability, and the like, and generally may be selected in a wide range from about 1 to about 1000 parts by weight per 1 part by weight of the substrate, and is preferably about 5 to about 500 parts by weight, more preferably from about 10 to about 100 parts by weight, and specifically preferably from about 15 to about 50 parts by weight.

The cyclization is preferably carried out by continuous feeding of a mixture of β-ketoamide-N-sulfonic acid represented by Formula (3) or a salt thereof with a solvent and a mixture of acid anhydride [sulfuric anhydride (SO₃) and the like] with a solvent into a tubular flow reactor or a motionless mixer able to be equipped with a cooler for cooling the reactor from the outside, such as a cooling jacket and a cooling tank (refrigerant tank). The reaction temperature of the cyclization may be set appropriately in consideration of reaction rate and the like.

As for the tubular reactor, a common stainless steel tube and a lined tube lined with glass or Teflon (registered trademark) or the like may be used, but the material is not limited to these materials. Furthermore, the inner diameter of the tube to be used is not specifically limited, but it is preferable that, in consideration of removal of reaction heat during the cyclization, the inner diameter is preferably several tens of mm or less (for example, from about 0.2 to about 30 mm) and specifically preferably 10 mm or less (for example, from about 0.2 to about 10 mm). Furthermore, the length of the tube is set so as to satisfy a residence time required for the reaction. The residence time is from about 0.001 to about 60 seconds, preferably from 0.01 to 40 seconds, and more preferably from 0.1 to 10 seconds (specifically from 1 to 10 seconds). The residence time (sec) is a value determined by the equation: [capacity of the reactor (ml)]/[total fed amount of raw material mixture (ml/sec)].

The tubular reactor may be equipped with an apparatus for accelerating a mixing of β-ketoamide-N-sulfonic acid represented by Formula (3) or a salt thereof with the acid anhydride [sulfuric anhydride (SO₃) and the like], at an inlet part of the tubular reactor. Examples of the apparatus are stirring mixers, ultrasonic mixers, motionless mixers such as a static mixer, piping joints, and the like (hereinafter, sometimes simply referred to as “premixer”). When the premixer is equipped at the inlet part of the tubular reactor, the residence time in the premixer is for example from about 0.0005 to about 30 seconds, preferably from about 0.01 to about 20 seconds, and more preferably from about 0.1 to about 10 seconds (specifically from about 1 to about 10 seconds), and the subsequent residence time in the tubular reactor is for example from about 0.001 to about 60 seconds, preferably from about 0.01 to about 40 seconds, and more preferably from about 0.1 to about 30 seconds (specifically from about 1 to about 30 seconds).

Furthermore, a motionless mixer such as a static mixer may also be used as the reactor. When the motionless mixer is used as the reactor, a motionless mixer with a larger inner diameter than that of the tubular reactor may be used since the motionless mixer may remove the reaction heat sufficiently. For example, the inner diameter of the motionless mixer is from about 0.2 to about 30 mm, and preferably from about 0.5 to about 20 mm. A type of the motionless mixer is not specifically limited, but as a typical motionless mixer, a Sulzer static mixer, a Kenics static mixer and the like may be used. When the motionless mixer is used as the reactor, the residence time is for example from about 0.001 to about 60 seconds, preferably from about 0.01 to about 40 seconds, and more preferably from about 0.03 to 10 seconds. In this case, such premixer described above may also be equipped at the inlet part of the motionless mixer. In this case, the residence time in the premixer is for example from about 0.0005 to about 30 seconds, preferably from about 0.01 to about 20 seconds, and more preferably from about 0.1 to about 10 seconds (specifically from about 1 to about 10 seconds), and the subsequent residence time in the motionless mixer is for example from about 0.001 to about 60 seconds, preferably from about 0.01 to about 40 seconds, and more preferably from about 0.03 to about 10 seconds.

The number of elements in the static mixer is not specifically limited, but is for example 5 or more (from about 5 to about 25), and preferably 10 or more.

By the above mentioned cyclization, generally, elimination of water or a base [for example, in the case that a salt of the compound represented by Formula (3) is used as the substrate] derives the 3,4-dihydro-1,2,3-oxathiazin-4-one-2,2-dioxide compound represented by Formula (1). In this case, under some conditions of the amount of the acid anhydride [sulfuric anhydride (SO₃) and the like] used, an adduct of the 3,4-dihydro-1,2,3-oxathiazin-4-one-2,2-dioxide compound represented by Formula (1) and the acid anhydride [sulfuric anhydride (SO₃) and the like] or the like is formed. In this case, after the cyclization, subsequent hydrolysis of the adduct or the like may derive the 3,4-dihydro-1,2,3-oxathiazin-4-one-2,2-dioxide compound represented by Formula (1).

The hydrolysis of the reaction mixture obtained by the cyclization is carried out by, for example, mixing the reaction mixture with water or a solution containing water (for example, an aqueous solution of sulfuric acid). If required, the hydrolysis is carried out after a suitable treatment of the reaction mixture. The hydrolysis may be carried out by any system such as continuous system, batch system, or semi-batch system. In the case of the continuous hydrolysis, a stirring tank is used and the continuous reactor used for the cyclization may be used. The temperature of water or the solution containing water for the hydrolysis and the hydrolysis reaction temperature are for example from 0 to 50° C., and preferably from 10 to 40° C. Furthermore, the amount of water (or the amount of water contained in the solution containing water) is for example from about 1 to about 100 mol, preferably from about 1 to about 50 mol, and more preferably from about 2 to about 20 mol per 1 mol of the acid anhydride used for the cyclization. Water may be used in large excess. The reaction time of the hydrolysis (in the case of continuous system, residence time) is for example 1 hour or less (from about 0.1 minute to about 1 hour), and preferably from about 1 to about 10 minutes.

Together with the formation of 3,4-dihydro-1,2,3-oxathiazin-4-one-2,2-dioxide compound represented by Formula (1) by the hydrolysis, a hydrolysate of the acid anhydride is formed as a by-product. In the case that sulfuric anhydride (SO₃) is used as the acid anhydride, sulfuric acid is formed as the by-product. The formed 3,4-dihydro-1,2,3-oxathiazin-4-one-2,2-dioxide compound represented by Formula (1) may be separated and purified by a separation means such as washing, liquid separation, concentration, solvent exchange, extraction, crystallization, recrystallization, and column chromatography. For example, the compound represented by Formula (1) may be isolated by the following procedures; the reaction mixture after the completion of the hydrolysis is separated into a liquid in organic phase containing the compound represented by Formula (1) and an aqueous phase (an aqueous solution of sulfuric acid and the like), the liquid in organic phase is washed with water or a solution containing water (for example, an aqueous solution of sulfuric acid), and then operations such as concentration, solvent exchange, and crystallization are carried out. Water or an aqueous solution of sulfuric acid or the like may be used for the crystallization solvent. Furthermore, the compound represented by Formula (1) remaining in the aqueous phase may be extracted and collected by adding a solvent incompatible (or immiscible) with water [the solvent used for the cyclization or esters of an organic monocarboxylic acid or organic dicarboxylic acid (for example, the esters listed in the description of the reaction solvent) and the like] to the aqueous phase. The 3,4-dihydro-1,2,3-oxathiazin-4-one-2,2-dioxide compound represented by Formula (1) obtained in this manner generally contains, as an impurity, sulfuric acid derived as the by-product of the hydrolysis or derived from the aqueous solution of sulfuric acid used as the crystallization solvent. In the invention, the compound represented by Formula (1) containing such sulfuric acid as the impurity is used as the material. The amount of sulfuric acid contained as the impurity is not specifically limited, and the material with a sulfuric acid content to the compound represented by Formula (1) of, for example, 100% by weight or less (from about 0.1 to about 100% by weight), preferably 50% by weight or less (from about 0.1 to about 50% by weight), and more preferably 20% by weight or less (from about 0.1 to 20% by weight) is suitably used.

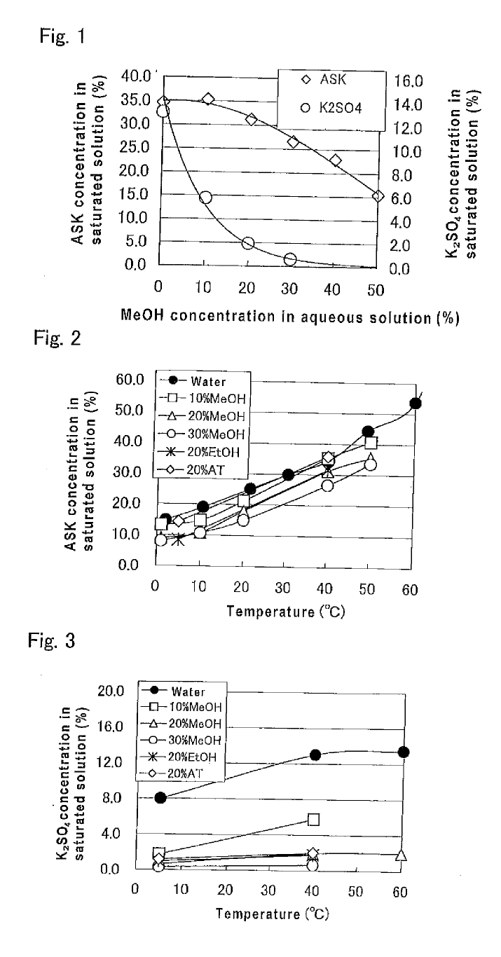

An important feature of the producing method of the invention is: (i) neutralizing a compound represented by Formula (1) with potassium hydroxide in a mixed solvent of water and a water soluble organic solvent to obtain a potassium salt of 3,4-dihydro-1,2,3-oxathiazin-4-one-2,2-dioxide compound represented by Formula (2) (in a dissolving state), and precipitating potassium sulfate formed as a by-product during the neutralization (first producing method); or (ii) neutralizing a compound represented by Formula (1) with potassium hydroxide in a solvent containing at least water to obtain a potassium salt of 3,4-dihydro-1,2,3-oxathiazin-4-one-2,2-dioxide compound represented by Formula (2) (in a dissolving state), and, after the neutralization, adding a water soluble organic solvent into the aqueous solution containing the potassium salt represented by Formula (2) to precipitate potassium sulfate formed as a by-product during the neutralization (second producing method). In a mixed solvent of water and a water soluble organic solvent, the difference in the solubility between the compound represented by Formula (2) and potassium sulfate is large, so that potassium sulfate is crystallized first. Thus, the compound represented by Formula (2) with an extremely low content of potassium sulfate may be obtained from the solution after the removal of the precipitated potassium sulfate. When water or a water soluble organic solvent such as methanol is used alone, the difference in the solubility between the compound represented by Formula (2) and potassium sulfate is not so large, so that the compound represented by Formula (2) and potassium sulfate cannot be separated efficiently by the crystallization operation using these solvents as the crystallization solvent. FIG. 1 shows the experimental result of the relations between a amount of methanol/water in a water-methanol mixed solvent (methanol concentration (%)) and solubilities of potassium salt of 6-methyl-3,4-dihydro-1,2,3-oxathiazin-4-one-2,2-dioxide (ASK) and of potassium sulfate (K₂SO₄), at 40° C. in the solvent (concentration (%) in saturated solution) Furthermore, FIG. 2 shows the experimental result of the relations between solubilities of ASK in various solvents (concentration (%) in saturated solution) and temperature, and FIG. 3 shows the experimental result of the relations between solubilities of potassium sulfate (K₂SO₄) in various solvents (concentration (%) in saturated solution and temperature. In Figures, MeOH is methanol, EtOH is ethanol, AT is acetone, and % is % by weight.

As for the water soluble organic solvent, any organic solvent soluble in water may be used, but solvents in which the solubility of the compound represented by Formula (2) is not too low, for example, alcohols such as methanol, ethanol, and isopropyl alcohol; and ketones such as acetone are preferably used. Among them, methanol is specifically preferred.

The ratio of water to a water soluble organic solvent used in the precipitation of potassium sulfate is, in the first producing method, the ratio of water to a water soluble organic solvent in the mixed solvent used in the neutralization, and, in the second producing method, the ratio of water to a water soluble organic solvent in the aqueous solution used in the addition of a water soluble organic solvent into the aqueous solution containing the potassium salt represented by Formula (2) after the neutralization. The ratio of water/water soluble organic solvent (weight ratio) is, for example, from about 1/99 to about 99/1, preferably from about 20/80 to about 95/5, more preferably from about 40/60 to about 93/7, and specifically preferably from about 65/35 to about 90/10. If the ratio of water is too low, not only the solubility of potassium sulfate but also that of the compound represented by Formula (2) are decreased, so that the amount of the solvent to be used is increased as well as the solubility difference is decreased, and thus separation efficiency is also apt to be deteriorated. Furthermore, if the ratio of water is too high, the solubility difference between each other is decreased, and thus the separation efficiency is apt to be deteriorated.

Introduction methods of the compound represented by Formula (1), water, a water soluble organic solvent and potassium hydroxide into the system are not specifically limited. For example, the compound represented by Formula (1) and potassium hydroxide may be introduced into the system in the solid state or in the dissolved state in a solvent such as water, a water soluble organic solvent or a mixture of these solvents. In the introduction method of the water soluble organic solvent, for example, the water soluble organic solvent is added to a solution of potassium hydroxide, and then the mixture may be introduced into a solution containing the compound represented by Formula (1); the water soluble organic solvent is added to a solution containing the compound represented by Formula (1), and then the mixture may be introduced into a solution of potassium hydroxide; or the water soluble organic solvent may be added and introduced into a solution containing potassium hydroxide and the compound represented by Formula (1). Furthermore, for example, in the second producing method of the invention, the following method may be used: an organic solvent solution (a methylene chloride solution and the like) of the compound represented by Formula (1) and an aqueous solution of potassium hydroxide are mixed in two different immiscible liquids system for neutralization, an aqueous phase containing the potassium salt represented by Formula (2) and an organic solvent phase are separated, and then an water soluble organic solvent is added to the aqueous phase to precipitate potassium sulfate formed as a by-product during the neutralization.

The amount of potassium hydroxide used for the neutralization is any amount sufficient to change the compound represented by Formula (1) to the potassium salt, and is, for example, from about 1 to about 3 mol, preferably from about 1 to about 1.5 mol, and more preferably from about 1 to about 1.1 mol per 1 mol of the total amount of the compound represented by Formula (1) and sulfuric acid contained as the impurity. If the amount of potassium hydroxide is too small, the neutralization is not completed, and if the amount of potassium hydroxide is too large, by-products are formed and the quality of the target compound is apt to be deteriorated.

The temperature during the neutralization and the precipitation of potassium sulfate may be not higher than the boiling point of an used solvent, and is generally from about 0 to about 100° C., preferably from about 5 to about 80° C., and more preferably from about 10 to about 60° C. If the temperature is too low, the difference in solubility between the compound represented by Formula (2) and potassium sulfate becomes small, so that the separation efficiency is apt to be deteriorated. If the temperature is too high, it becomes energetically-disadvantageous.

Potassium sulfate precipitated by the operation is removed by solid-liquid separation such as filtration and centrifugation. From the viewpoint of operability and the like, it is preferred that potassium sulfate is removed by filtration. The compound represented by Formula (2) may be isolated from the solution after the removal of potassium sulfate (filtrate and the like) by, for example, crystallization. The crystallization may be carried out by concentrating and/or cooling the solution after the removal of potassium sulfate. The precipitated compound represented by Formula (2) may be obtained by solid-liquid separation (filtration, centrifugation and the like). The purity of the compound represented by Formula (2) obtained in this manner may be increased by further recrystallizing. As for the recrystallization solvent, for example, water may be used.

After the crystallization, the residual solution (filtrate and the like) obtained by the solid-liquid separation of the precipitated compound represented by Formula (2) may be recycled to the neutralization step. Even if the solution is recycled repeatedly, the quality of the compound represented by Formula (2) is kept. Furthermore, in the case that the crystallization is carried out by the concentration, the distillate may be recycled to the neutralizing step. Furthermore, in the case that the recrystallization is carried out, the solution after the separation of the precipitated target compound (filtrate and the like) may be recycled to the neutralization step.

The compound represented by Formula (2) obtained in this manner has an extremely low content of potassium sulfate, a high purity, and a high quality, so that it may he used as sweeteners or raw materials therefor in food industry or intermediate materials in fine chemicals or the like. Specifically, the compound of Formula (2) wherein R¹ is methyl group and R² is hydrogen atom (potassium salt of 6-methyl-3,4-dihydro-1,2,3-oxathiazin-4-one-2,2-dioxide) is used as the sweetener [Acesulfame (Acesulfame K)] in the food industry, so that it is specifically useful.

### Examples

The present invention will be described in further detail with reference to several examples below, which are not intended to limit the scope of the invention.

### Example 1

Distilled water and methanol were added to a wet crystal of 6-methyl-3,4-dihydro-1,2,3-oxathiazin-4-one-2,2-dioxide containing sulfuric acid as an impurity, and then, the wet crystal was dissolved in the added distilled water and methanol. The solution was composed of 60.4 g of 6-methyl-3,4-dihydro-1,2,3-oxathizin-4-one-2,2-dioxide, 5.3 g of sulfuric acid, 282.7 g of water, and 99.0 g of methanol. To the solution, 49.3 g of an aqueous solution of potassium hydroxide at 50% by weight was added dropwise under stirring with removal of the reaction heat to neutralize at 35° C. A precipitate was formed slightly in the reaction mixture. The reaction mixture was filtrated at 40° C. to remove the precipitate. The precipitate contained 7.06 g of potassium sulfate. The filtrate contained 2.4 g of potassium sulfate.

### Comparative Example 1

Distilled water was added to a wet crystal of 6-methyl-3,4-dihydro-1,2,3-oxathiazin-4-one-2,2-dioxide containing sulfuric acid as an impurity, and then, the wet crystal was dissolved in the distilled water. The solution is composed of 60.7 g of 6-methyl-3,4-dihydro-1,2,3-oxathiazin-4-one-2,2-dioxide, 5.3 g of sulfuric acid, and 383.7 g of water. To the solution, 49.1 g of an aqueous solution of potassium hydroxide at 50% by weight was added dropwise under stirring with removal of the reaction heat to neutralize at 35° C. A precipitate was formed slightly in the reaction mixture. The reaction mixture was filtrated at 40° C. to remove the precipitate. The precipitate did not contain potassium sulfate. The filtrate contained 9.6 g of potassium sulfate.

### INDUSTRIAL APPLICABILITY

As for potassium salts of 3,4-dihydro-1,2,3-oxathiazin-4-one-2,2-dioxide compounds useful as sweeteners or raw materials therefor in food industry or intermediate materials for fine chemicals and the like, a method for producing high quality potassium salts of 3,4-dihydro-1,2,3-oxathiazin-4-one-2,2-dioxide compounds in which a contamination of potassium sulfate which causes a quality deterioration may be controlled with an extremely small amount may be provided.

1. A method for producing a potassium salt of 3,4-dihydro-1,2,3-oxathiazin-4-one-2,2-dioxide compound represented by the following formula (2) 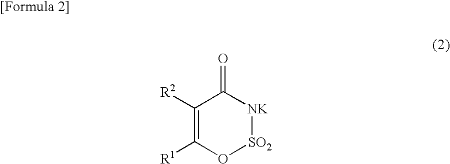
(wherein R¹ and R² are the same as or different from each other and are hydrogen atom or an organic group inert to the reaction)
and containing sulfuric acid as an impurity, the method comprising steps of:
neutralizing 3,4-dihydro-1,2,3-oxathiazin-4-one-2,2dioxide compound represented by the following formula (1) 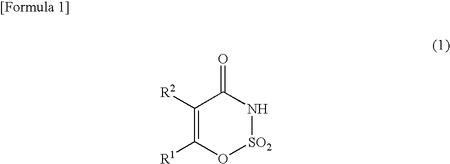
(wherein R¹ and R² are as defined above) with potassium hydroxide in a mixed solvent of water and a water soluble organic solvent to obtain a solution containing said potassium salt; and
precipitating potassium sulfate derived from the sulfuric acid upon taking the step of neutralizing.

2. A method for producing a potassium salt of 3,4-dihydro-1,2,3-oxathiazin-4-one-2,2-dioxide compound represented by the following formula (2) 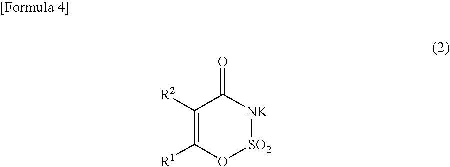
(wherein R¹ and R² are the same as or different from each other and are hydrogen atom or an organic group inert to the reaction)
and containing sulfuric acid as an impurity, the method comprising steps of:
neutralizing 3,4-dihydro-1,2,3-oxathiazin-4-one-2,2-dioxide compound represented by the following formula (1) 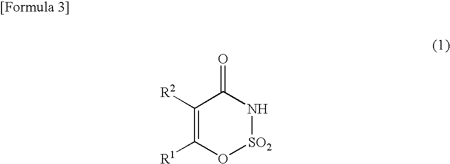
(wherein R¹ and R² are as defined above) with potassium hydroxide in a solvent containing at least water to obtain an aqueous solution containing said potassium salt; and
precipitating potassium sulfate derived from the sulfuric acid by adding a water soluble organic solvent into the aqueous solution containing said potassium salt after the step of neutralizing.

3. The method for producing the potassium salt of 3,4-dihydro-1,2,3-oxathiazin-4-one-2,2-dioxide compound of claim 1 or 2, wherein the water soluble organic solvent is methanol.

4. The method for producing the potassium salt of 3,4-dihydro-1,2,3-oxathiazin-4-one-2,2-dioxide compound of claim 1 or 2, further comprising a step of separating and removing the precipitated potassium sulfate by filtration.

5. The method for producing the potassium salt of 3,4-dihydro-1,2,3-oxathiazin-4-one2,2-dioxide compound of claim 1 or 2, further comprising a step of crystallizing said potassium salt from the solution after removing the precipitated potassium sulfate.

6. The method for producing the potassium salt of 3,4-dihydro-1,2,3-oxathiazin-4-one-2,2-dioxide compound of claim 5, further comprising a step of recycling the solution after separating and removing the crystallized potassium salt of 3,4-dihydro-1,2,3-oxathiazin-4-one-2,2-dioxide compound to the step of neutralizing.
